(19) United States
(12) Patent Application Publication
(10) Pub. No.: US 2021/0267654 A1
(43) Pub. Date: Sep. 2, 2021

(54) Module For A Modular Bone Plate, Arranged To Receive An Anchoring Device, Modular Bone Plate Of This Construction, And System Comprising Said Plate And Anchoring Device

(71) Applicants: Arnaud DESTAINVILLE, SAINT-XANDRE (FR); Olivier RICHART, LA ROCHELLE (FR)

(72) Inventors: Arnaud DESTAINVILLE, SAINT-XANDRE (FR); Olivier RICHART, LA ROCHELLE (FR)

(21) Appl. No.: 17/253,908

(22) PCT Filed: Jun. 7, 2019
(86) PCT No.: PCT/FR2019/051380

§ 371 Date: Dec. 18, 2020

(30) Foreign Application Priority Data
Jun. 19, 2018 (FR) 1855375

(51) Int. Cl. A61B 17/80

(52) U.S. Cl. CPC A61B 17/8023; A61B 17/8061; A61B 17/8052

(57) ABSTRACT

The invention relates to a module (100) for a modular bone plate, comprising a male coupling portion (102), a female coupling portion (104) and an intermediate body (106) that extends between said male and female coupling portions, the male coupling portion, or female coupling portion respectively, being arranged to cooperate with a female portion, or male portion respectively, of a second module of a modular bone plate, the male coupling portion and the female coupling portion having recesses (1024, 1026) to form a hole, referred to as an assembly hole, which passes from an upper surface to a lower surface of the module and is arranged to cooperate with a bone anchoring device that has two threads spaced apart by a non-threaded zone, wherein one of the two threads is intended to anchor the bone device in a bone and the assembly hole is partially threaded so as to cooperate with the other of the two threads of said anchoring device.

### TECHNICAL FIELD

The present invention relates to a module for a modular bone plate, arranged to receive an anchoring device.

It also relates to a modular bone plate comprising a module according to the invention, and to a system comprising the plate and the anchoring device.

The field of the invention is that of bone plates which are suitable for being used to achieve osteosynthesis. The field of the invention is more particularly that of modular bone plates for the treatment of fractures or osteotomies that affect the skeletal system.

### PRIOR ART

The technical solutions that are used the most today for treating fractures or osteotomies in human or veterinary orthopedic and trauma surgery consist in a restraint system that combines a plate and screws, usually metal, which are intended to be anchored in the bone or bones in question. This system is thus intended to be fixed to healthy and/or damaged bone surfaces, in order to ensure the stability of said regions and the compression of bone fragment, ultimately making it possible to reconstruct and strengthen the bone in question (long bone, cranial bone, spinal bone, etc.).

Said plates, referred to as “compression”, are thus positioned against the periosteal surface of the bones to be treated, the design and dimensions thereof being closely linked to two factors: on the one hand the bone anatomy of the patient, and on the other hand the type and location of the fracture or the osteotomy.

Indeed, there is significant inter- and intra-populational anatomical variability of the skeleton, making it necessary to define widely dimensioned intervals, as well as dedicated designs for restraint systems, in order to respond to all the situations encountered by the practitioners. It is also proposed that the surgeon should himself perform the anatomical deformation of the proposed plates, in order to optimize the contact with the bone. However, this practice is barely satisfactory, since, often imprecise and time-consuming, it is a hazardous situation during a surgical intervention, as well as for postoperative recoveries.

Moreover, the design variability must integrate the high number of types of skeletal fractures listed (long bones, spinal bones, simple or complex fracture, extra-articular or intra-articular, transverse, multi-fragmentary, etc.), as well as osteotomies which are, by nature, induced.

Today, the result of all these requirements, increased by varied options for production materials, is that an extremely large number of osteosynthesis devices have been made available to human or veterinary orthopedic and trauma surgeons. It is therefore not infrequent that a practitioner must select, during the intervention, from among several dozens of plates and screws, in order to perform the intervention, which is associated with a plurality of instruments specially designed for fitting the associated implants. In addition to the time that the practitioner must take to refine his preoperative analysis in order to determine the references he needs from the dozens proposed, it is necessary to include, upstream and downstream, the unwieldy management of this stock, whether this be the financial and administrative impact, or the management of cleaning and sterilization of said devices. Owing to the historical choice of the very large supply in order to meet all the needs of orthopedic and trauma practitioners, the operational impact for the health facilities is inextricably linked to the analysis.

The modular nature of osteosynthesis systems, and more particularly of plates, is thus a solution for better meeting the expectations of practitioners from the surgical perspective, but must also make it possible, by extension and by a suitable and competitive manufacturing process, and simple and attractive surgical instruments, to be integrated in a wider offering which must also optimize the operational impact within health facilities and medical teams.

The U.S. Pat. No. 8,343,154 proposes forming a modular bone plate to overcome this drawback at least in part. A module for a modular bone plate is described therein, comprising a male engagement coupling portion, a female engagement coupling portion, and an intermediate body that extends between the male coupling portion and the female coupling portion, the intermediate body comprising a lower contact surface for contact with the bone, and an upper surface that is opposite the surface for contact with the bone. The engagement of one portion on the other brings about the locking of the two modules. More precisely, the engagement between a female portion and a male portion of two modules is achieved in accordance with a plane substantially in parallel with the median plane of the two portions.

It will be understood that any force perpendicular to the median plane of the two portions tends to deform, and potentially, in the case of intense force, dismantle, said two portions. Furthermore, it will be understood that the manufacture of such modules, and their manipulation, is not easy.

The patent EP 270 629, emerging from the international application WO 2012172517, aims to solve these problems, and proposes a new module for a modular bone plate. A module for a modular bone plate is described therein, comprising a male coupling portion, a female coupling portion, and an intermediate body that extends between the male coupling portion and the female coupling portion, the intermediate body comprising a lower contact surface for contact with the bone, and an upper surface that is opposite the surface for contact with the bone.

Nonetheless, the device proposed by said patent is not completely satisfactory.

Indeed, said device is still complicated to produce. Furthermore, the proposed solution requires systematic recourse to a third family of components, in order to ensure the stability of the compression screws in the plate (nuts, screw plugs, etc.). The third family of components does not have the effect of locking a first module onto a second module, but merely of preventing the compression screw from coming out of the bone into which it is screwed. The need for a third family of components to be present opposes the desired simplification for the practitioners.

The object of the invention is that of proposing a solution that aims to overcome the above-mentioned disadvantages, at least in part.

### DISCLOSURE OF THE INVENTION

This object is achieved by a module for a modular bone plate, comprising a male coupling portion, a female coupling portion, and an intermediate body that extends between the male coupling portion and the female coupling portion, the intermediate body comprising a lower surface that is arranged so as to be in contact with a bone, and an upper surface that is opposite the lower surface.

The male or female portion is arranged so as to cooperate with a female or male portion, respectively, of a second module for a modular bone plate according to the invention.

The module comprises a lower surface which is arranged so as to be in contact with the bone, and an upper surface which is opposite the lower surface.

Furthermore, the male portion and the female portion comprise recesses which form a hole, referred to as the assembly hole, when the female coupling portion receives a male coupling portion of a second module for a bone plate.

The assembly hole is a through-hole, passing from the upper surface of the module as far as the lower surface of the module, and

According to the invention, the assembly hole is arranged so as to cooperate with a bone anchoring device comprising two threads, preferably having different pitches, which are spaced apart, or not, by a non-threaded region, one of the two threads being provided for anchoring the bone device in a bone, and the assembly hole is furthermore threaded at least in part, the thread of the assembly hole being arranged so as to cooperate with the other of the two threads of the anchoring device.

More generally, the bone anchoring device may comprise two pitches, preferably having different pitches, which are spaced apart, or not, by a non-threaded region, one of the two pitches being provided for anchoring the bone device in a bone, and the assembly hole is furthermore threaded at least in part, the thread of the assembly hole being arranged so as to cooperate with the other of the two pitches of the anchoring device.

Typically, the anchoring device may be a screw, made of a metal material, preferably medical grade, of the stainless steel, titanium, or titanium alloy type, of the TA6V type.

The thread, or more generally the pitch, provided to cooperate with the thread of the assembly hole, may comprise a double screw thread, so as to accelerate the locking.

The internal portions of the male and/or female coupling portions are formed of a core made of a metal material, preferably medical grade, of the stainless steel, titanium, or titanium alloy type, of the TA6V type.

The lower and upper surfaces of the male coupling portions and of the intermediate body are made of polymer, for example polyether ether ketone (PEEK) or polylactic acid (PLA).

The thread preferably extends at least over a perimeter of the recess of the female coupling portion.

The thread may extend over a perimeter of the male coupling portion, at least in part.

According to one option, the assembly hole may comprise a spherical portion which provides a seat for a spherical head of a bone anchor.

According to another option, the assembly hole may comprise a tapered portion which provides a seat for a tapered head of a bone anchor.

According to one option, the assembly hole may comprise a tapered portion, for example for receiving a tapered head or a spherical head.

According to one option, the module may comprise at least one additional hole which may extend from the upper surface of the module as far as the lower surface of the module. The additional hole may be equidistant from the assembly holes.

The at least one additional hole may be threaded at least in part and/or may comprise a spherical portion which provides a seat for a spherical head of a bone anchor, comprise a tapered portion which provides a seat for a tapered head of the bone anchor.

According to one option, the additional hole may comprise a tapered portion, for example for receiving a tapered head or a spherical head.

According to one option, the male coupling portion has a tenon and the female coupling portion has a mortise.

The assembly hole may pass through the tenon of the male coupling portion and the mortise of the female coupling portion. Advantageously, the thread may extend axially over a perimeter of the mortise, at least in part.

According to one embodiment, the male coupling portion may comprise a double tenon and the female coupling portion may comprise a double mortise, the assembly hole passing through each of the tenons of the double tenon and each of the mortises of the double mortise when the male coupling portion receives a female coupling portion of a second module for the bone plate.

The thread may thus extend axially over a perimeter of each of the two tenons of the male coupling portion, at least in part.

The thread may thus extend axially over a perimeter of each of the two mortises of the female coupling portion, at least in part.

According to a second aspect of the invention, an end module is proposed for a modular bone plate, comprising a coupling portion, male or female, and an intermediate body extending from the coupling portion, the end module comprising a lower surface which is arranged so as to be in contact with a bone, and an upper surface which is opposite the lower surface, said coupling portion being arranged so as to cooperate with a coupling portion of another module according to the first aspect of the invention, or one or more of the improvements thereof, or another end module. The end module according to the invention comprises a recess.

When the coupling portion of the end module is coupled to the other module, the respective recesses of the coupling portion of said end module and the coupling portion of said other module form a hole, referred to as an assembly hole.

The assembly hole is a through-hole, passing from the upper surface of the module as far as the lower surface of the module.

Furthermore, the end module may comprise, on the side opposite the coupling portion thereof, an end having a non-aggressive cross section, preferably rounded, in order to avoid trauma to the surrounding soft tissue at the time of implantation of the end module.

When the end module comprises a male coupling portion, the male coupling portion may comprise a tenon, and when said end module comprises a female coupling portion, the female coupling portion may comprise a mortise, it being possible for the female coupling portion to comprise a lower surface which is arranged so as to be in contact with the bone, and an upper surface which is opposite the lower surface.

The assembly hole may pass through the tenon of the male coupling portion and the mortise of the female coupling portion.

According to a third aspect of the invention, a modular bone plate is proposed comprising a first and a second module for a modular bone plate according to the first or the second aspect of the invention, or one or more of the improvements thereof.

According to a fourth aspect of the invention, a system is proposed comprising a modular bone plate according to the third aspect of the invention, or one or more of the improvements thereof, and a bone anchoring device. The bone anchoring device may be a screw device.

By way of example, the bone anchoring device may comprise two threads, preferably having different pitches, which are spaced apart by a non-threaded region, one of the two threads being provided for anchoring the bone device in a bone, the assembly hole of the module being threaded, at least in part, in order to cooperate with the other thread.

### DESCRIPTION OF THE FIGURES

Other data, features and advantages of the present invention will emerge from reading the description of implementations and embodiments, which are in no way limiting, with reference to the accompanying drawings, in which:

FIG. 1a and FIG. 1b are, respectively, perspective views of a first embodiment of a modular bone plate according to the invention, in an exploded view and assembled, respectively,

FIG. 2a and FIG. 2b are, respectively, perspective views of a first embodiment of a module according to the invention, having a length that is equal to one unit of length according to the invention, from a rear, or female, face, and from a front, or male, face, respectively,

FIG. 2c shows a first module according to a first variant of the first embodiment of a module according to the invention, having a length that is equal to 2 units of length according to the invention,

FIG. 2d shows a second module according to the first variant of the first embodiment of a module according to the invention, having a length that is equal to 4 units of length according to the invention,

FIG. 2e shows a third module according to the first variant of the first embodiment of a module according to the invention, having a length that is equal to 6 units of length according to the invention,

FIG. 2g and FIG. 2h are, respectively, perspective views of a second variant of the first embodiment of a module according to the invention, having a length that is equal to one unit of length according to the invention, from a rear, or female, face, and from a front, or male, face, respectively,

FIG. 3 is a plan view of the module shown in FIG. 2, in which two dotted lines are shown which separate, virtually, the functions of the module,

FIG. 4 is a cross-sectional view of the module shown in FIG. 2, according to the sectional plane A-A shown in FIG. 3,

FIG. 5 is a cross-sectional view of the module shown in FIG. 2, according to the sectional plane C-C shown in FIG. 4,

FIG. 6 is a cross-sectional view of the module shown in FIG. 2, according to the sectional plane E-E shown in FIG. 4,

FIG. 7 is a partial perspective view of the bone plate shown in FIG. 1, provided with various screws,

FIG. 8 is a plan view of the bone plate shown in FIG. 7,

FIG. 9 has a left-hand part which is a cross-sectional view of the bone plate shown in FIG. 7, according to the sectional plane A-A shown in FIG. 8, and a right-hand part showing the detail B shown in the left-hand part of FIG. 9,

FIG. 10 is a perspective view of a second embodiment of a modular bone plate according to the invention,

FIG. 11 is a perspective view of a second embodiment of a module according to the invention,

FIG. 12 is a vertical and longitudinal cross-sectional view of the module shown in FIG. 11,

FIGS. 13, 14 and 15 show various embodiments of the plates according to the invention.

### DESCRIPTION OF EMBODIMENTS

Since the embodiments described in the following are in no way limiting, it is in particular possible to envisage variants of the invention that comprise only a selection of features described in the following, in a manner isolated from the other features described, if this selection of features is sufficient for providing a technical advantage or for distinguishing the invention from the prior art. This selection comprises at least one feature, preferably functional and without structural details, or having some of the structural details if this part alone is sufficient for providing a technical advantage or for distinguishing the invention from the prior art.

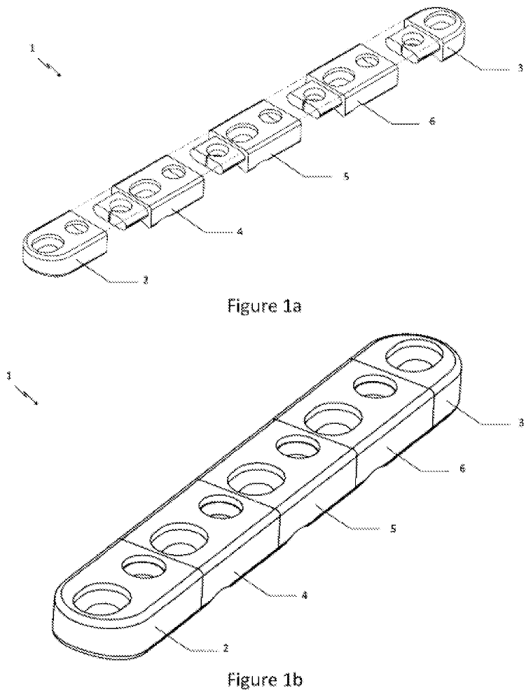

FIG. 1a is an exploded view, in the longitudinal direction, of a first embodiment of a modular bone plate 1 according to the invention, extending in a longitudinal direction.

FIG. 1b is an assembled view, in the longitudinal direction, of the first embodiment of the modular bone plate 1 according to the invention, extending in the longitudinal direction.

The modular bone plate 1 comprises two end parts—an end module 2 according to the invention and an end module 3 according to the invention, respectively—which are interconnected by a plurality of modules according to the invention, numbered 4, 5 and 6, respectively, according to the embodiment shown.

Each of the modules 4, 5 and 6 has a longitudinal direction which is substantially identical to the longitudinal direction of the bone plate.

In the embodiment shown, the modules 2 and 3 also have a longitudinal direction which is substantially identical to the longitudinal direction of the bone plate.

In the embodiment shown, the modules 4, 5 and 6 are identical.

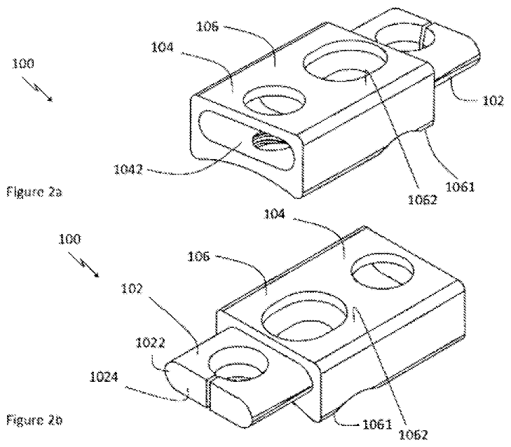

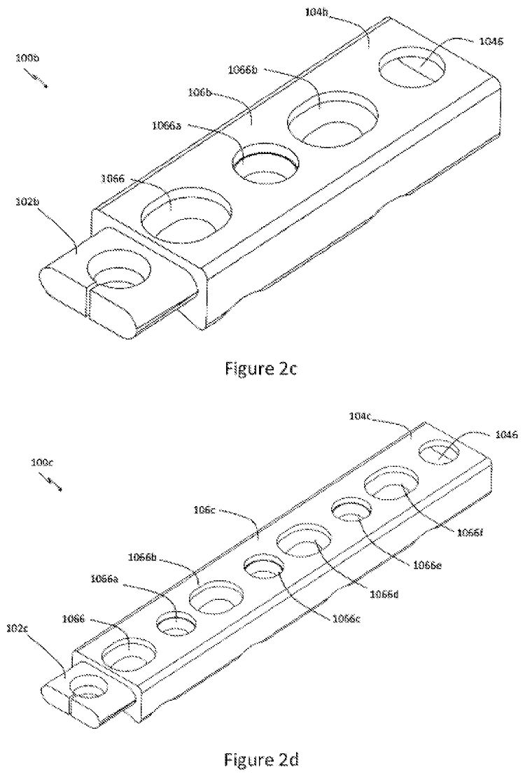

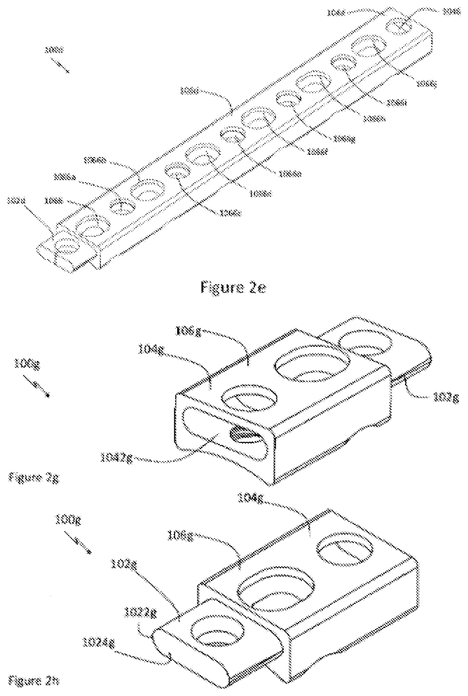

FIG. 2a is a view, from a rear, or female, face, of a first embodiment of a module 100 according to the invention, having a length that is equal to one unit of length according to the invention.

FIG. 2b is a view, from a front, or male, face, of the first embodiment of the module 100.

In terms of function, the module 100 comprises a male coupling portion 102, a female coupling portion 104, and an intermediate body 106 that extends between the male coupling portion 102 and the female coupling portion 104.

As can be seen in FIG. 1, the male coupling portions of the modules 5 and 6 are arranged so as to cooperate with the female coupling portions of the modules 4 and 5.

Likewise, the end module 2, referred to as proximal, comprises a female coupling portion which is arranged so as to cooperate with the male coupling portion of the module 4.

In addition, the end module 3, referred to as terminal, comprises a male coupling portion which is arranged so as to cooperate with the female coupling portion of the module 6.

Structurally, the intermediate body 104 is generally paralepidid in shape, preferably rectangular paralepidid.

The intermediate body 106 comprises a lower surface 1061, also referred to as an internal surface, or surface positioned opposite the bone surface, which lower surface is arranged so as to be in contact with a bone, and an upper surface 1062, also referred to as an external surface, or surface furthest from the bone surface, opposite the lower surface 1061.

The female coupling portion comprises a lower surface which is arranged so as to be in contact with the bone, which portion is an extension of the lower surface 1061 of the intermediate body, and an upper surface which is opposite the lower surface and is an extension of the upper surface 1062 of the intermediate body.

The male coupling portion 102 has a tenon 1022 extending axially, with respect to the intermediate body 106, from the side opposite the female coupling portion 104.

According to the embodiment shown, the tenon 1022 comprises a free end 1024 which is oblong in shape, and is generally cylindrical oblong in shape, having a generatrix in parallel with the longitudinal direction of the module 100.

The female coupling portion 104 has a mortise 1042 extending axially, with respect to the intermediate body 106, from the side opposite the female coupling portion 102.

According to the embodiment shown, the base of the mortise 1042 also has an oblong surface. The mortise is generally cylindrical oblong in shape, having a generatrix in parallel with the longitudinal direction of the module 100.

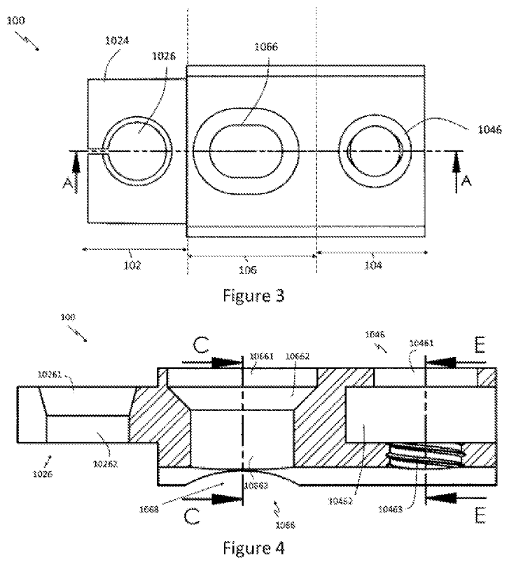

As can be seen more clearly in FIG. 3, the male coupling portion 102 and the female coupling portion 104 comprise recesses or bores, 1026 and 1046, respectively.

In the embodiment shown, the tenon comprises a slit that is perpendicular to the plane of FIG. 3 and extends over the entire height of the tenon 1022, and longitudinally, from the external part of the tenon 1022 located on the side opposite the intermediate body 106, as far as the recess 1026.

When the female coupling portion of one module receives a male coupling portion of a second module, the respective recesses of the female coupling portion of the module and the male coupling portion of the second module form a hole, referred to as a locking hole.

The intermediate body 106 furthermore comprises an additional hole 1066, which is oblong in this embodiment, extending from the upper surface 1061 of the intermediate body as far as the lower surface 1062 of the intermediate body.

The additional hole makes it possible to receive compression screws. In the present description, “compression screw” means a bone anchoring system, the purpose of which is to bring two or more bone fragments closer together.

FIG. 4, which is a vertical cross section along the longitudinal axis of the module 100, shows the geometry of the recesses.

The recess 1026 formed in the male coupling portion 102, and the tenon 1022 thereof, has a smooth surface which comprises, vertically, from the upper surface as far as the lower surface, a tapered portion 10261 and a cylindrical portion 10262 which are centered according to a vertical axis.

The tapered portion 10261 has a diameter which decreases from an external diameter in the region of the upper surface of the male coupling portion, as far as an internal diameter.

The cylindrical portion 10262 has a diameter which is equal to the internal diameter of the tapered portion 10261.

The recess 1046 formed in the female coupling portion 104 comprises, vertically from the upper surface of the female coupling portion 104 as far as the lower surface of the female coupling portion 104: a first cylindrical portion 10461, a second portion 10462 having a cylindrical shaping, and a third cylindrical portion 10463 comprising a thread, all the portions being centered according to a vertical axis.

The first cylindrical portion 10461 is smooth and has a diameter which is equal to the external diameter of the tapered portion 10261.

The second cylindrical portion 10462 is smooth and forms the mortise 1042 of the female coupling portion 104. Thus, the dimensions of the second cylindrical portion are the same as those of the tenon 1022 of the male coupling portion 102.

More precisely, the length of the tenon is equal to, or slightly less than, the depth of the mortise, the width of the tenon is equal to the length of the mortise, the thickness of the tenon is equal to, or slightly less than, the width of the mortise, the respective lengths of the cheeks of the tenon and of the mortise being equal.

The third cylindrical portion 10463 has a diameter that is substantially equal to that of the diameter of the cylindrical portion 10262.

The thread of the third cylindrical portion 10463 makes it possible to receive a screw thread of a screw that corresponds to said thread.

When a male portion of one module is coupled to a female portion of another module, the assembly hole can receive the above-mentioned screw. When the screw is screwed into the female portion, the assembly hole is thus a locking hole for the male and female portions of the two modules.

The additional hole 1066 comprises, from the upper surface of the intermediate body 106 as far as the lower surface of the intermediate body 106, a first cylinder 10661 comprising an oblong base, a tapered portion 10662, and a second cylinder 10663.

The first cylinder 10661 is smooth and has an oblong base.

The tapered portion 10662 is smooth and has a diameter that decreases from a first diameter on the side of the external surface 1061 of the intermediate body 106 as far as a second diameter of the side of the internal surface 1062 of the intermediate body 106.

The second cylinder 10662 is smooth and has a diameter which is equal to the second diameter of the tapered portion 10662.

Furthermore, a recess 1068 is visible, which has a cross section in the shape of circular arc.

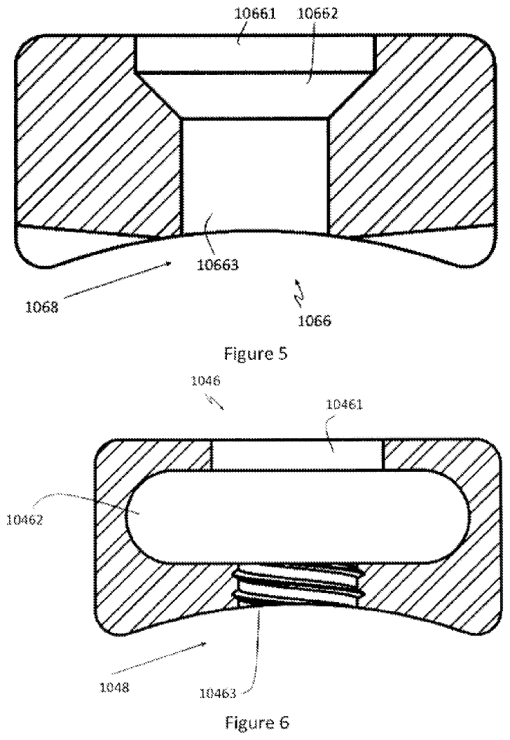

FIG. 5 is a cross-sectional view of the module 100, according to the sectional plane C-C shown in FIG. 4. Furthermore, it can be seen that the recess 1068 still has a cross section in the shape of circular arc in this plane. Within the context of the use of a module 100 for a modular plate for a long bone, the function of said recess is to fit the surface of the long bone as well as possible.

FIG. 6 is a cross-sectional view of the module 100, according to the sectional plane E-E shown in FIG. 4. This cross section shows the shape of the mortise 1042, in a plane transverse to the longitudinal direction, which shape corresponds to that of the tenon 1022. Furthermore, a recess 1048 is also visible, which has a cross section in the shape of circular arc in this plane. The function of this recess 1048 is to limit the contact of the module 100 with the periosteal bone tissue, which contact, if extended, can result in states of tissue and vascular necrosis.

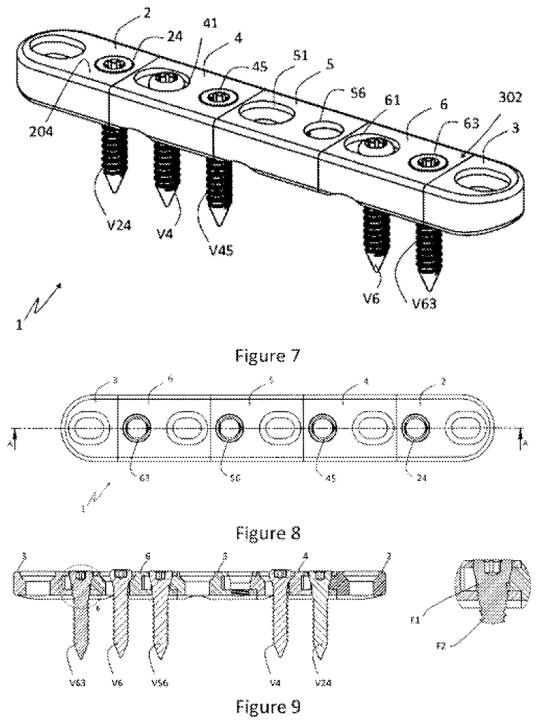

As is shown by FIG. 7, the locking holes 45 and 56, formed, respectively, by the female coupling portion of the module 4 and the male coupling portion of the module 5, the female coupling portion of the module 5 and the male coupling portion of the module 6, are through-holes, passing from the upper surface of the female coupling portion and as far as the lower surface of the female coupling portion.

As has been seen, the assembly holes 45 and 56 pass through the tenon of the male coupling portion and the mortise of the female coupling portion.

The additional holes 41, 51, and 61, formed in the intermediate bodies of the modules 4, 5, and 6, respectively, are through-holes, passing from the upper surface of the intermediate body as far as the lower surface of the intermediate body.

The end module 2 comprises a female coupling portion 204 which is arranged so as to cooperate with the male coupling portion of the diaphyseal module 4.

The end module 3 comprises a male coupling portion 302 which is arranged so as to cooperate with the male coupling portion of the diaphyseal module 6.

The female portion of the end module 2 comprises a recess and, when the coupling portion of the end module 2 is coupled to the coupling portion of the diaphyseal module 4, the respective recesses of the coupling portions of the end module 2 and of the diaphyseal module 4 form a hole 24, referred to as an end assembly hole.

The end assembly hole 24 is a through-hole, passing from the upper surface of the female coupling portion as far as the lower surface of the female coupling portion of the end module 2.

The end assembly hole 24 passes through the tenon of the male coupling portion of the diaphyseal module 4, and the mortise of the female coupling portion of the end module 2.

The male portion of the end module 3 comprises a recess and, when the coupling portion of the end module 3 is coupled to the coupling portion of the diaphyseal module 6, the respective recesses of the coupling portions of the end module 3 and of the diaphyseal module 6 form a hole 63, referred to as an end assembly hole.

The end assembly hole 63 is a through-hole, passing from the upper surface of the female coupling portion as far as the lower surface of the female coupling portion of the diaphyseal module 6.

The end assembly hole 63 passes through the mortise of the female coupling portion of the diaphyseal module 6, and the tenon of the male coupling portion of the end module 3.

FIG. 7 also shows three bone anchoring devices, in the form of screws V24, V45, V63, which are inserted in the locking holes 24, 45 and 63, respectively, and comprise a thread which is provided for anchoring the bone device in a bone.

Other screws, V4 and V6, respectively, which are also provided for achieving bone anchoring, are shown in FIG. 7, passing through the additional holes 41 and 61, respectively.

FIG. 7 shows neither anchoring means nor locking means passing through the assembly hole 56. However, during use the assembly hole 56 must be provided at least with a locking means for the diaphyseal modules 5 and 6.

FIG. 8 is both a plan view of the elements shown in FIG. 1b, and a plan view of FIG. 7, from which the screws have been masked.

The left-hand part of FIG. 9 is a cross section according to the plane A-A shown in FIG. 8, the plane A-A being the vertical plane according to the longitudinal direction.

The right-hand part of FIG. 9 shows the detail B, denoted on the left-hand part of FIG. 9.

According to a first variant of the first embodiment of the module 100, described only with respect to the differences thereof from the first embodiment of the module 100, a diaphyseal module may be of a different length.

FIG. 2c shows a first module 100b according to the first variant, also comprising a male coupling portion 102b, a female coupling portion 104b, and an intermediate body 106b that extends between the male coupling portion 102b and the female coupling portion 104b.

As before, the intermediate body comprises the additional hole 1066 and the recess 1046.

The module 100b differs from the module 100 only in that it comprises two additional holes 1066a and 1066b which are arranged longitudinally on the intermediate body 106b.

The additional hole 1066a has a geometry which matches that described with reference to the assembly hole of the module 100.

The additional hole 1066b has a geometry which matches that described with reference to the additional hole 1066 of the module 100.

The module 100b has a length that is equal to 2 units of length according to the invention.

FIG. 2d shows a second module 100c according to the first variant.

The module 100c again comprises a male coupling portion 102c, a female coupling portion 104c, and an intermediate body 106c that extends between the male coupling portion 102c and the female coupling portion 104c.

As before, the intermediate body 106c comprises the additional hole 1066 and the recess 1046.

The module 100c differs from the module 100b only in that it comprises four additional holes 1066c, 1066d, 1066e and 1066f which are arranged longitudinally on the intermediate body 106c.

The three additional holes 1066a, 1066c and 1066e have a geometry which matches that described with reference to the assembly hole 1066a of the module 100b.

The three additional holes 1066b, 1066d and 1066f have a geometry which matches that described with reference to the additional hole 1066b of the module 100b.

The module 100c also has a length that is equal to 4 units of length according to the invention.

FIG. 2e shows a third module 100d according to the first variant.

The module 100d again comprises a male coupling portion 102d, a female coupling portion 104d, and an intermediate body 106d that extends between the male coupling portion 102d and the female coupling portion 104d.

As before, the intermediate body 106d comprises the additional hole 1066 and the recess 1046.

The module 100d differs from the module 100c only in that it comprises four additional holes 1066g, 1066h, 1066i and 1066j which are arranged longitudinally on the intermediate body 106c.

The two additional holes 1066g and 1066i have a geometry which matches that described with reference to the assembly hole 1066a of the module 100c.

The additional holes 1066h and 1066j have a geometry which matches that described with reference to the additional hole 1066b of the module 100c.

The module 100d also has a length that is equal to 6 units of length according to the invention.

According to a second variant of the first embodiment of the module 100, described only with respect to the differences thereof from the first embodiment of the module 100, but optionally able to be combined with the first variant of the first embodiment of the module 100, the tenon may possibly not comprise the slit described above.

FIG. 2g is a view, from a rear, or female, face, of a module 100g according to the second variant, having a length that is equal to one unit of length according to the invention.

FIG. 2h is a view, from a front, or male, face, of the module 100g.

The module 100g also comprises a male coupling portion 102g, a female coupling portion 104g, and an intermediate body 106g that extends between the male coupling portion 102g and the female coupling portion 104g.

The male coupling portion 102g comprises a tenon 1022g extending axially, with respect to the intermediate body 106g, from the side opposite the female coupling portion 104g.

According to the embodiment shown, the tenon 1022g comprises a free end 1024g which is oblong in shape, and is generally cylindrical oblong in shape, having a generatrix in parallel with the longitudinal direction of the module 100g.

The female coupling portion 104g comprises a mortise 1042g extending axially, with respect to the intermediate body 106g, from the side opposite the female coupling portion 102g.

According to the embodiment shown, the base of the mortise 1042g also comprises an oblong surface. The mortise is generally cylindrical oblong in shape, having a generatrix in parallel with the longitudinal direction of the module 100g.

In the embodiment shown, the tenon 1022g does not comprise the slit described above.

According to a third variant of the first embodiment of the module 100, described only with respect to the differences thereof from the first embodiment of the module 100, but optionally able to be combined with the first and/or the second variant of the first embodiment of the module 100, the third portion 10463 of the recess 1046 may be tapered and, preferably, comprise a thread.

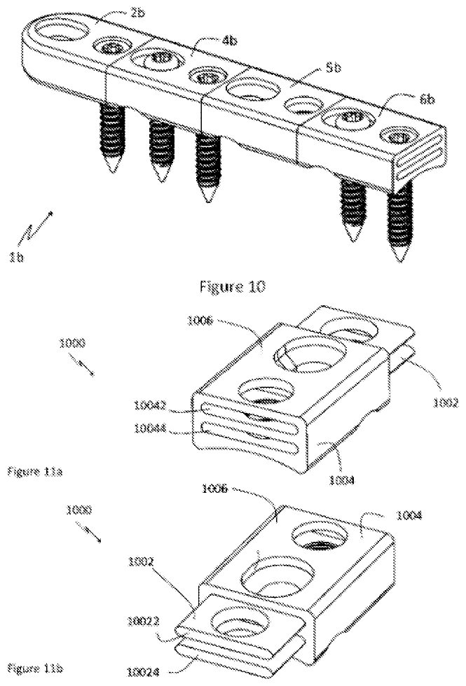

FIG. 10 is a perspective view of a second embodiment of a modular bone plate 1b according to the invention, described only with respect to the differences thereof from the first embodiment of a bone plate according to the invention.

The figure shows an end portion which is an end module 2b according to the invention, and a plurality of modules according to the invention, numbered 4b, 5b and 6b, respectively.

Each of the modules 4b, 5b and 6b has a longitudinal direction which is substantially identical to the longitudinal direction of the bone plate.

In the embodiment shown, the modules 4b, 5b and 6b are identical.

The three diaphyseal modules 4b, 5b and 6b are identical to a second embodiment of a module according to the invention.

The module 1000 is in accordance with a second embodiment of a module according to the invention.

FIG. 11 is a view, from a rear, or female, face, of a module 1000 having a length that is equal to one unit of length according to the invention.

FIG. 11b is a view of the module 1000 from a front, or male, face.

The module 1000 comprises a male coupling portion 1002, a female coupling portion 1004, and an intermediate body 1006 that extends between the male coupling portion 1002 and the female coupling portion 1004

The male coupling portion 1002 comprises a double tenon 10022, 10024 extending axially, with respect to the intermediate body 1006, from the side opposite the female coupling portion 1004.

According to the embodiment shown, each of the tenons 10022, 10024 has an oblong free end. The female coupling portion 1004 comprises a double mortise 10042 and 10044 extending axially, with respect to the intermediate body 1006, from the side opposite the female coupling portion 1002.

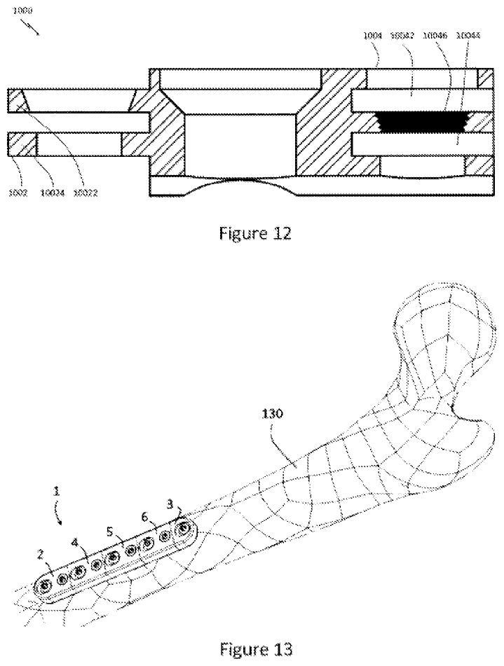

As shown in FIG. 12, which is a vertical and longitudinal cross-sectional view of the module shown in FIG. 11, the female coupling portion 1004 comprises a thread which is arranged between the two mortises.

According to a first variant of the second embodiment of the module 1000, described only with respect to the differences thereof from the second embodiment of the module 1000, the free end of at least one of the tenons is oblong in shape.

FIG. 13 is a perspective view of a diaphyseal and proximal portion of a femur 130 on which the plate 1, described above, is arranged.

It will be understood that the plate 1 is straight and is preferably used for treating diaphyseal fractures of long bones, such as the femur, the tibia, the radius, the ulna, and the humerus.

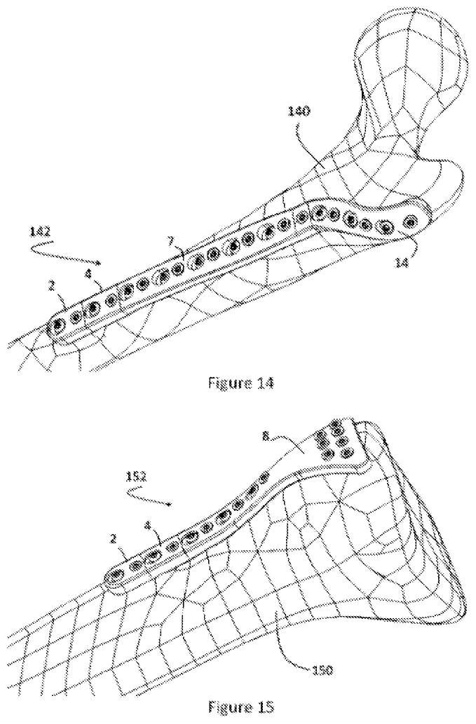

FIG. 14 is a perspective view of a portion of a femur 140 on which a module plate 142 according to the invention is arranged.

The plate 142 is formed of the end module 2, the diaphyseal module 4, an intermediate module 7, and an end module 14.

The intermediate module 7 matches a module according to the first or the second embodiment of a module according to the invention, or one or more of the variants thereof.

The end module 14 matches an end module according to the first or the second embodiment of an end module according to the invention, or one or more of the variants thereof.

Ideally, the intermediate module 7 has an anatomical shape that is matched to the diaphyseal and metaphyseal shape of the proximal portion of the femur 140, and serves as a connection between the diaphyseal 4 and the end module 14.

The end module 14 has a shape which is matched to the trochanteric anatomy of the epiphyseal proximal portion of the femur 140.

FIG. 15 is a perspective view of a proximal portion of a tibia 150 on which a module plate 152 according to the invention is arranged.

The plate 15 is formed of the end module 2, the diaphyseal module 4, and an end module 8.

The end module 8 matches an end module according to the first or the second embodiment of an end module according to the invention, or one or more of the variants thereof.

The end module 8 has a shape which is matched to the anatomy of the internal zone of the epiphyseal proximal portion of the tibia 150.

Of course, the invention is not limited to the embodiments described above, and a number of developments can be made to said embodiments, without departing from the scope of the invention. Moreover, the various features, types, variants, and embodiments of the invention may be associated with one another, in accordance with various combinations, insofar as they are not mutually incompatible or exclusive.

Furthermore, the number of diaphyseal modules which may be implemented on a modular plate is unlimited.

In particular, a modular plate may be formed just of two end modules.

1. A module for a modular bone plate, having a male coupling portion, a female coupling portion, and an intermediate body that extends between the male coupling portion and the female coupling portion,
the module comprising a lower surface which is arranged so as to be in contact with a bone, and an upper surface which is opposite the lower surface,
said male coupling portion or female coupling portion is arranged so as to cooperate with a female or male portion, respectively, of a second module for a modular bone plate,
the male coupling portion and the female coupling portion comprising recesses, and,
when the female coupling portion receives a male coupling portion of a second module for a bone plate, the respective recesses of the female coupling portion of the module and the male coupling portion of the second module form a hole, referred to as an assembly hole,
said assembly hole being a through-hole, passing from the upper surface of the module as far as the lower surface of said module,
characterized in that
the assembly hole is arranged so as to cooperate with a bone anchoring device having two threads, optionally having different pitches, which are spaced apart by a non-threaded region, one of the two threads being provided for anchoring the bone device in a bone, and
the assembly hole is furthermore threaded at least in part, the thread of the assembly hole being arranged so as to cooperate with the other of the two threads of the anchoring device.

2. The module according to claim 1, wherein the thread extends at least over a perimeter of the recess of the female coupling portion.

3. The module according to claim 1, wherein the thread extends at least over a perimeter of the recess of the male coupling portion.

4. The module according to claim 1, wherein the assembly hole comprises a tapered portion which provides a seat for a tapered head of the bone anchor.

5. The module according to claim 1, comprising at least one additional hole which extends from the upper surface of the module as far as the lower surface of the module.

6. The module according to claim 1, wherein the at least one additional hole is threaded at least in part and/or comprises a tapered portion which provides a seat for a tapered head of the bone anchor.

7. The module according to claim 1, wherein the male coupling portion has a tenon and the female coupling portion has a mortise,
the assembly hole passing through the tenon of the male coupling portion and the mortise of the female coupling portion.

8. The module according to claim 7, wherein the thread extends axially over a perimeter of the mortise, at least in part.

9. The module according to claim 1, wherein the male coupling portion has a double tenon and the female coupling portion has a double mortise, the assembly hole passing through each of the tenons of the double tenon and each of the mortises of the double mortise when the male coupling portion receives a female coupling portion of a second module for the bone plate.

10. The module according to claim 9, wherein the thread extends axially over a perimeter of each of the two tenons of the male coupling portion, at least in part.

11. The module according to claim 9, wherein the thread extends axially over a perimeter of each of the two mortises of the female coupling portion, at least in part.

12. An end module for a modular bone plate, having a coupling portion, which is male or female, and an intermediate body that extends between from the coupling portion,
the end module comprising a lower surface which is arranged so as to be in contact with a bone, and an upper surface which is opposite the lower surface, said coupling portion being arranged so as to cooperate with a coupling portion of another module or another end module, said end module comprising a recess, and
when the coupling portion of the end module is coupled to the other module or end module, the respective recesses of the coupling portion of said end module and the coupling portion of said other module or end module form a hole, referred to as an assembly hole,
said assembly hole being a through-hole, passing from the upper surface of the module or end module as far as the lower surface of the module or end module,
the assembly hole being arranged so as to cooperate with a bone anchoring device having two threads, optionally having different pitches, which are spaced apart by a non-threaded region, one of the two threads being provided for anchoring the bone device in a bone, and
the assembly hole being threaded at least in part, the thread of the assembly hole being arranged so as to cooperate with the other of the two threads of the anchoring device.

13. A modular bone plate comprising at least a first and a second module and/or end module for a modular bone plate.

14. A system comprising at least two modules and/or end modules for a modular bone plate according to claim 13, and a bone anchoring device.

15. The system according to claim 14, wherein the bone anchoring device has two threads, optionally having different pitches, which are spaced apart by a non-threaded region, one of the two threads being provided for anchoring the bone device in a bone, the assembly hole of the module being threaded, at least in part, in order to cooperate with the other thread.

16. The module according to claim 4, comprising at least one additional hole which extends from the upper surface of the module as far as the lower surface of the module.

17. The module according to claim 4, wherein the male coupling portion has a tenon and the female coupling portion has a mortise, the assembly hole passing through the tenon of the male coupling portion and the mortise of the female coupling portion.

18. The module according to claim 16, wherein the male coupling portion has a tenon and the female coupling portion has a mortise,
the assembly hole passing through the tenon of the male coupling portion and the mortise of the female coupling portion.

19. The module according to claim 1, wherein the male coupling portion has a double tenon and the female coupling portion has a double mortise, the assembly hole passing through each of the tenons of the double tenon and each of the mortises of the double mortise when the male coupling portion receives a female coupling portion of a second module for the bone plate.

20. The module according to claim 16, wherein the male coupling portion has a double tenon and the female coupling portion has a double mortise, the assembly hole passing through each of the tenons of the double tenon and each of the mortises of the double mortise when the male coupling portion receives a female coupling portion of a second module for the bone plate.
